(19) United States
(12) Patent Application Publication
(10) Pub. No.: US 2021/0128862 A1
(43) Pub. Date: May 6, 2021

(54) CUSHION WITH BUCKLING PREVENTION STRUCTURE

(71) Applicant: KONINKLIJKE PHILIPS N.V., EINDHOVEN (NL)

(72) Inventors: ROBERT WILLIAM BAIKO, JR., PITTSBURGH, PA (US); LAUREN PATRICIA CHODKOWSKI, PITTSBURGH, PA (US)

(21) Appl. No.: 16/472,471

(22) PCT Filed: Dec. 21, 2017
(86) PCT No.: PCT/EP2017/083968

§ 371 Date: Jun. 21, 2019

Related U.S. Application Data
(60) Provisional application No. 62/438,499, filed on Dec. 23, 2016.

(51) Int. Cl. A61M 16/06

(52) U.S. Cl. CPC A61M 16/0622; A61M 16/0666; A61M 16/0611

(57) ABSTRACT

A cushion for use in a system providing a flow of breathing gas to the airway of a patient includes a first end having a sealing flap structured to sealingly engage a surface of the patient about an airway of the patient and a second end adapted to receive the flow of breathing gas. A wall portion extends between the first end and the second end and defines a passage which extends between the first end and the second end and which is structured to communicate the flow of breathing gas from the second end to the first end. The wall portion includes a notch which is positioned and structured to deform in a predetermined manner such that an angle which the wall portion makes with a longitudinal axis of the passage increases from a first value to a second value upon deformation of the notch.

### CROSS-REFERENCE TO RELATED APPLICATIONS

This patent application claims the priority benefit under 35 U.S.C. § 119(e) of U.S. Provisional Application No. 62/438,499 filed on Dec. 23, 2016, the contents of which are herein incorporated by reference.

### BACKGROUND OF THE INVENTION

### 1. Field of the Invention

The present invention generally relates to a cushion for use on a patient interface device in a pressure support system that supplies a flow of gas to the airway of a patient, and, more particularly, to a cushion in which at least a portion of the cushion includes a structure which prevents buckling in a sealing portion of the cushion. The present invention also relates to a system for delivering a flow of breathing gas to the airway of a patient which includes such a cushion.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NW). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort in order to prevent leaks. Buckling of sealing flaps in cushions is the leading cause of leak experienced by CPAP masks. When a buckle forms, a leak path is created which propagates to the exterior environment. The leak, depending on the location, can cause compliance issues because the leak can result in treatment gas blowing into the patient's eyes, across the patient's face, or toward a bed partner.

### SUMMARY OF THE INVENTION

Accordingly, it is an object of the present invention to provide an improved cushion for use in a patient interface device that overcomes the shortcomings of conventional cushions. As an aspect of the invention, a cushion for use in a system providing a flow of breathing gas to the airway of a patient is provided. The cushion comprises: a first end portion having a sealing flap which is structured to sealingly engage a surface of the patient about an airway of the patient; a second end portion adapted to receive the flow of breathing gas; and a wall portion which extends between the first end portion and the second end portion and defines a passage which extends between the first portion and the second portion. The passage is structured to communicate the flow of breathing gas from the second end portion to the first end portion. The wall portion includes a notch which is positioned and structured to deform in a predetermined manner such that an angle which the wall portion makes with a longitudinal axis of the passage increases from a first value to a second value upon deformation of the notch.

The notch may extend into the wall portion from an outer surface of the wall portion disposed opposite the passage. The wall portion may comprise an inner surface facing the passage, and the inner surface may be generally uninterrupted and smooth. Deformation of the notch may produce a tensioning force in at least a portion of the sealing flap. The notch may be disposed generally parallel to the sealing flap or generally perpendicular to the sealing flap and the wall portion may further comprise a second notch disposed generally parallel to the sealing flap or generally perpendicular to the sealing flap. The notch may be structured to deform from a first position in which the notch has a first opening width to a second position in which the notch has a second opening width which is less than the first opening width or which is greater than the first opening width.

As another aspect of the invention, a cushion for use in a system providing a flow of breathing gas to the airway of a patient is provided. The cushion comprises a first end portion having a sealing flap which is structured to sealingly engage a surface of the patient about an airway of the patient, the sealing flap defining a patient opening therein. The cushion further comprises a second end portion adapted to receive the flow of breathing gas; and a wall portion extending between the first end portion and the second end portion and defining a passage extending between the first portion and the second portion, the passage being structured to communicate the flow of breathing gas from the second end portion to the first end portion. The wall portion includes a notch which is positioned and structured to deform in a predetermined manner which increases the perimeter of the patient opening.

As yet another aspect of the invention, a system for providing a pressurized flow of gas to the airway of a patient is provided. The system comprises: a pressure/flow generating system; and a cushion as otherwise described herein.

### BRIEF DESCRIPTION OF THE DRAWINGS

FIG. 1 is a front isometric view of a patient interface device according to the principles of the present invention shown (schematically) connected to a gas flow/pressure generating system to form a patient interface system;

FIG. 2 is a rear isometric view of the patient interface device of FIG. 1;

FIG. 3 is a side elevation view of the cushion of the patient interface device of FIGS. 1 and 2;

FIG. 4 is a rear elevation view of the cushion of the patient interface device of FIGS. 1 and 2;

FIG. 5A is a sectional view of a portion of the cushion of the patient interface device of FIGS. 1 and 2, taken along line 5-5 of FIGS. 3 and 4, showing the portion disposed in a first position;

FIG. 5B is another version of the sectional view of the portion of the cushion of FIG. 5A shown displaced in a second position resulting from a force applied to the patient side of the cushion;

FIG. 6 is a side elevation view of another cushion in accordance with an example embodiment of the present invention;

FIG. 7A is a sectional view of the cushion of FIG. 6, taken along line 7-7 of FIG. 6, showing the cushion disposed in a first position;

FIG. 7B is another version of the sectional view of the cushion of FIG. 7A shown displaced in a second position resulting from a force applied to the patient side of the cushion;

FIG. 8 is a side elevation view of another cushion in accordance with an example embodiment of the present invention;

FIG. 9 is an isometric view of a nasal pillow cushion in accordance with another example embodiment of the present invention;

FIG. 10A is a sectional view of a portion of the nasal pillow of FIG. 9, taken along line 10-10 of FIG. 9, showing the portion disposed in a first position;

FIG. 10B is another version of the sectional view of the portion of the nasal pillow of FIG. 10A shown displaced in a second position resulting from a force applied to the patient side of the pillow; and

FIGS. 11A-11C show examples of attributes of notches which may be varied in accordance with example embodiments of the present invention.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

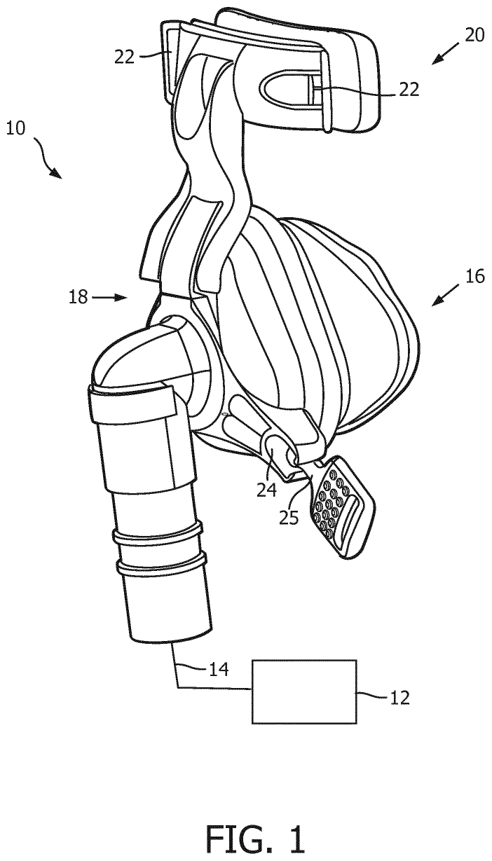

FIGS. 1-4 illustrate an exemplary embodiment of a patient interface device 10 and components thereof according to the principles of the present invention. Patient interface device 10 communicates a flow of breathing gas between the patient's airway and a pressure/flow generating system 12 (shown schematically), such as a ventilator, CPAP device, or variable pressure device, e.g., a BiPAP® device manufactured and distributed by Philips Respironics of Pittsburgh, PA, or an auto-titration pressure support system.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating system 12 is also referred to as a gas flow generating device, because flow results when a pressure gradient is generated. The present invention contemplates that pressure/flow generating system 12 is any conventional system for delivering a flow of gas to an airway of a patient or for elevating a pressure of gas at an airway of the patient, including the pressure support systems summarized above and non-invasive ventilation systems.

Communicating a flow of breathing gas between the patient's airway and pressure/flow generating system 12 includes delivering a flow of breathing gas to the patient from the pressure/flow generating device and exhausting a flow of gas from the patient to ambient atmosphere. The system for delivering a breathing gas to a patient according to the present invention comprises the pressure/flow generating system that produces a flow of gas, and a conduit 14, which is also referred to as a patient circuit, having a first end portion (not numbered) operatively coupled to the gas flow generating device and a second end portion (not numbered). Conduit 14 carries the flow of gas from pressure/flow generating device 12 during operation of the system to patient interface device 10, which is coupled to the second end portion of the conduit. Conduit 14 corresponds to any conduit suitable for communicating the flow of gas from the pressure/flow generating system to the patient interface device. A typical conduit is a flexible tube. A headgear assembly, which is not shown in the figures, attaches patient interface device 10 to the patient's head.

Patient interface device 10 includes a cushion, generally indicated at 16, and a mask shell 18 having a patient side and opposite thereto, an outer side. Attached to outer side of mask shell 18 is a conduit coupling member (not numbered) that couples mask shell 18 to conduit 14 so that a flow of gas is communicated to the interior of the patient interface device for subsequent delivery to the patient. Conversely, gas from the patient is communicated from the patient interface device into conduit 14, where an exhaust port is located. Mask shell 18 is preferably a generally rigid shell, and, in an exemplary embodiment of the present invention is formed from rigid plastic, such as polycarbonate. It is to be understood that the present invention contemplates that one or more of the size, shape, or composition of mask shell 18 may be varied without varying from the scope of the present invention. It is also to be understood that embodiments of the present concept may be employed in patient interface devices which do not utilize a mask shell.

In the illustrated embodiment of FIG. 1, mask shell 18 has a generally triangular shape having a forehead support portion 20 that includes headgear attaching elements in the form of receiving holes or slots 22 disposed on either side of forehead support portion 20 for receiving headgear straps (not illustrated). In the illustrated embodiment, the lower corners of mask shell 18 also include headgear attaching elements in the form of socket attachment elements 24, which cooperate with corresponding ball elements 25 on headgear straps (not illustrated). The ball and socket configuration, and other headgear attachment configurations suitable for use with the present invention, are disclosed, for example, without limitation, in commonly assigned U.S. Pat. No. 7,066,179, the contents of which are incorporated herein by reference.

It is to be understood that the present invention contemplates using any conventional connection assembly to attach a headgear or headgear strap to mask shell 18 or other suitable shell arrangement. It is to be further understood that the present invention also contemplates eliminating the forehead support entirely, so that the patient interface device is supported on the patient by cushion 16. If the forehead support is eliminated, a headgear attachment may be provided at the upper apex of the mask shell. The present invention also contemplates providing a post or other protrusion at the upper portion of the shell, i.e., the portion overlying the bridge of the nose, to which the headgear can be attached.

The present invention contemplates that the headgear suitable for use with patient interface device 10 is any conventional headgear used in the patient interface field. For example, without limitation, a typical headgear assembly comprises a headpiece that overlies a portion of the patient's crania and with headgear straps extending therefrom to adjustably connect the headgear to the mask.

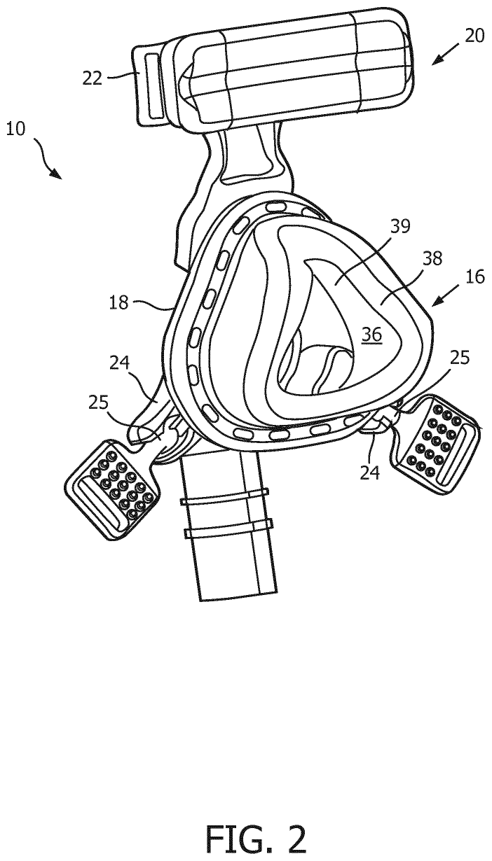

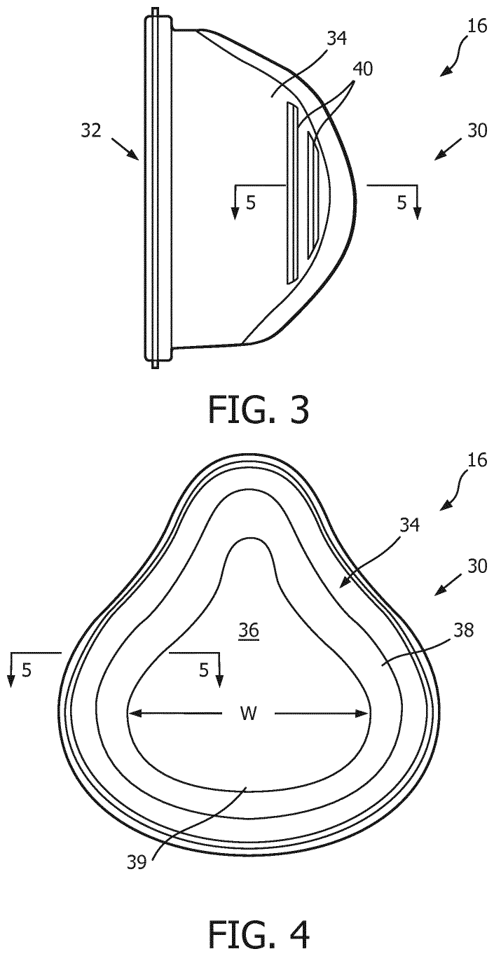

Referring to FIGS. 3 and 4, cushion 16 is preferably formed of a soft, cushiony, elastomeric material, such as silicone, appropriately soft thermoplastic elastomers, closed cell foam, thin materials, or any combination of suitable materials. Cushion 16 has a first end portion 30, a second end portion 32 disposed opposite first end portion 30, and a wall portion 34 extending between first end portion 30 and second end portion 32. A passage 36 is defined within cushion 16, generally by wall portion 34, which extends between first end portion 30 and second end portion 32 through which the flow of breathing gas produced by pressure/flow generating system 12 passes from second end portion 32 to first end portion 30 and then to the airway of the patient. First end portion 30 includes a sealing flap 38 which generally defines a patient opening 39 in first end portion 30. Sealing flap 38 is structured to sealingly engage a surface of a patient about an airway of the patient (e.g., flap 38 sealingly engages about a patient's nose, and thus sealingly engages about both nares of the patient). Second end portion 32 is adapted to receive the flow of breathing gas produced by pressure flow generating system 12 either via an intermediary component, such as mask 18 in FIGS. 1 and 2, or directly or indirectly via suitable arrangement.

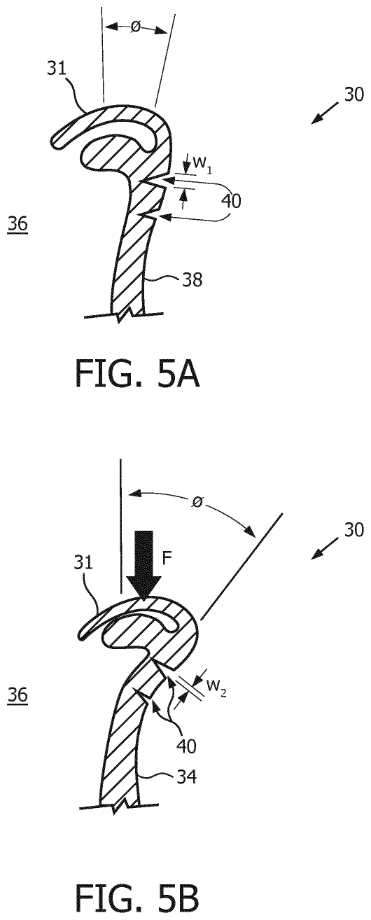

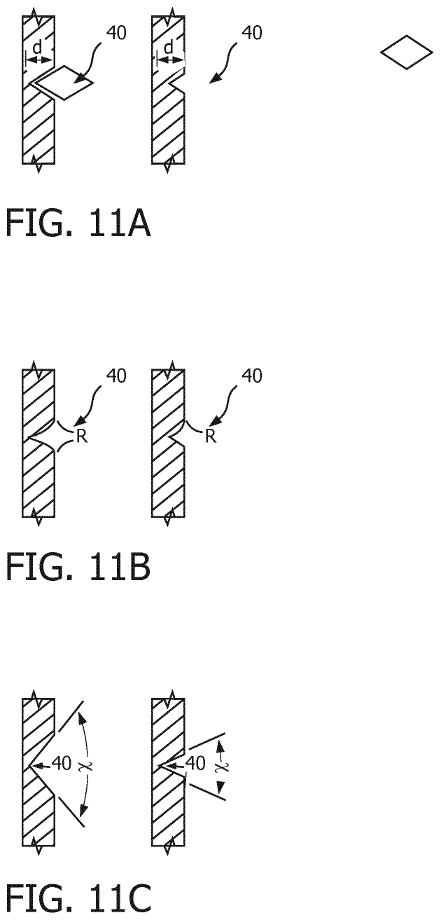

Referring to FIGS. 3, 5A and 5B, wall portion 34 includes a number of notches 40 which are each positioned and structured to deform in a predetermined manner upon application of a force F, such as when cushion 16 is pressed against the face of a patient. In general, each notch 40 is formed by the removal of material from the outside (i.e., the side disposed opposite and facing away from passage 36) of wall portion 34 without removing material from the inside (i.e., the side facing passage 36) of wall portion 34, thus leaving the sides of passage 36 (i.e., inner surface of wall portion 34) generally continuous, uninterrupted, and smooth (as there are no notches provided on the passage side of wall portion 34). It is to be appreciated that such “removal” may occur in the design stage and does not require that the material be removed by specific process during manufacture. In this fashion, each notch 40 will collapse causing the adjacent part of wall portion 34 to rotate and generally put end portion 30 in tension. Accordingly, such deformation of selected portions, or the entirety, of wall portion 34 results in outward (i.e., away from passage 36) movement of the region of wall portion 34 near first end portion 30, thus increasing one or more dimensions of patient opening 39 (e.g., without limitation, a width W). Such movement thus produces a tensioning force in at least a portion of sealing flap 38. Examples of various attributes (e.g., depth d, wall curvature R, and angle α) of notch 40 (or other notches described herein) which may be varied depending on a particular desired application are illustrated in FIGS. 11A-11C.

For example, in the example arrangement shown in FIGS. 3, 4, 5A and 5B, when compressed against the face of a patient (not shown), portions of wall portion 34 disposed generally on either side of the patient's nose deform outward generally as illustrated in moving from FIG. 5A to FIG. 5B such that an angle ϕ which wall portion makes with a longitudinal axis of passage 36 increases from a first value to a second value. During such movement, each of notches 40 generally collapse such that the width of the opening of each notch decreases from a first opening width w₁ to a lessor second opening width w₂. As a result of such collapse of notches 40, the sides of cushion 16 deform outward, causing width W of patient opening 39 to increase. At the same time, the other portions of the cushion 16 (i.e., generally the top and bottom of cushion 16 adjacent patient opening 39) are generally constrained due to their construction. As a result, the perimeter of patient opening 39 generally increases, thus producing a tensioning force in sealing flap 38 which removes/inhibits formation of undesirable buckles in sealing flap 38.

In the illustrated example, each of notches 40 are positioned generally parallel to at least a portion of first end portion 30 and/or sealing flap 38, however, it is to be appreciated that such notches 40 may be positioned in other orientations without varying from the scope of the present invention. It is also to be appreciated that the quantity of such notches 40 may also be varied without varying from the scope of the present invention.

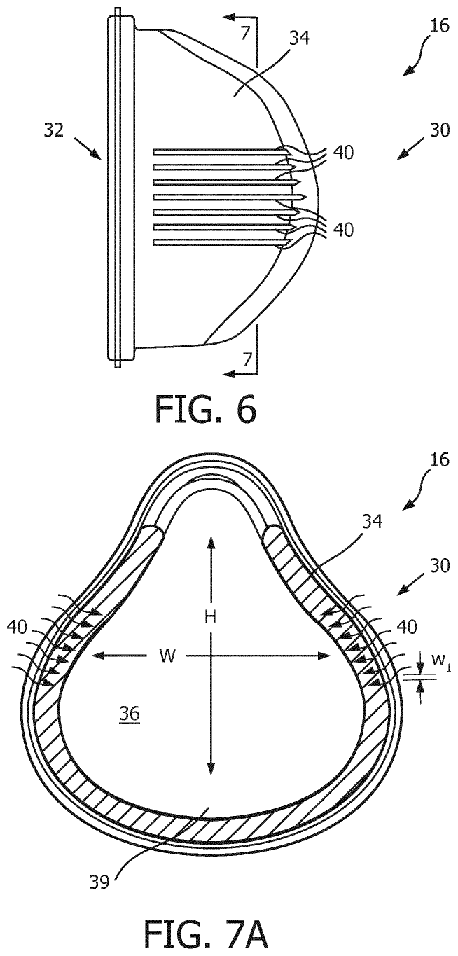

FIG. 6 shows a side elevation view of a cushion 16′ in accordance with another example embodiment of the present invention. Similar to cushion 16, cushion 16′ also includes a plurality of notches 40′. However, unlike cushion 16, notches 40′ are disposed generally perpendicular to first end portion 30 and/or sealing flap 38. Also, unlike notches 40 of cushion 16 which tend to collapse when cushion 16 is pressed against the face of a patient, notches 40′ tend to open when cushion 16′ is pressed against a face of a patient.

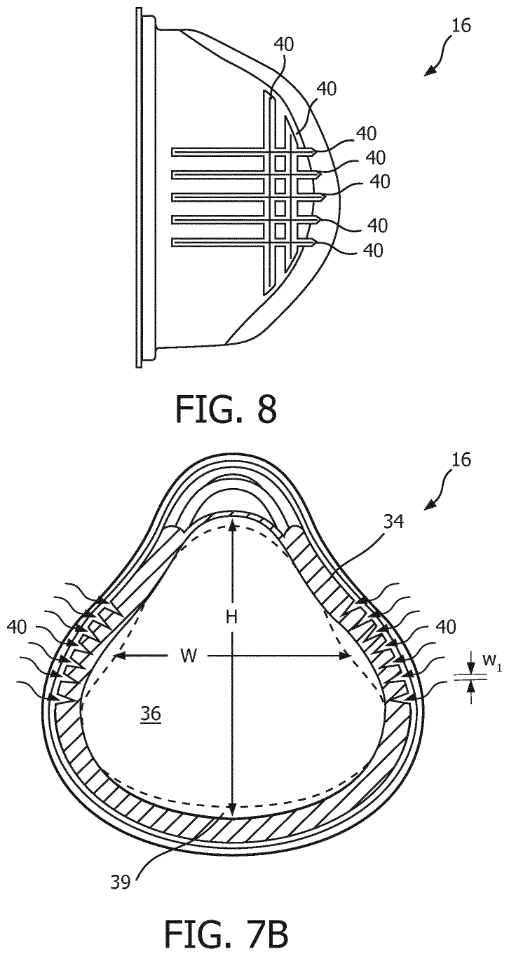

For example, in the example arrangement shown in FIGS. 6, 7A and 7B, when cushion 16′ is compressed against the face of a patient (not shown), portions of wall portion 34′ disposed generally on either side of the patient's nose deform generally outward in moving from FIG. 7A to FIG. 7B. During such movement, each of notches 40′ generally opens such that the width of the opening of each notch increases from a first opening width w₁ to a greater second opening width w₂. As a result of such expansion/opening of notches 40′, the sides of cushion 16′ deform outward, causing width W of patient opening 39 to increase (as shown by W′ in FIG. 7B). Also, as a result of such expansion of notches 40′ the height H of patient opening 39 may also increase (as shown by H′ in FIG. 7B). Such increase in the width W or height H of patient opening 39 causes the perimeter of patient opening 39 to increase, thus producing a tensioning force in sealing flap 38 which removes/inhibits formation of undesirable buckles in sealing flap 38.

FIG. 8 shows a side elevation view of a cushion 16″ in accordance with yet another example embodiment of the present invention. Cushion 16″ includes an intersecting combination of notches 40 and 40′ which provide for cushion 16″ for deform in ways similar to both of the examples previously described.

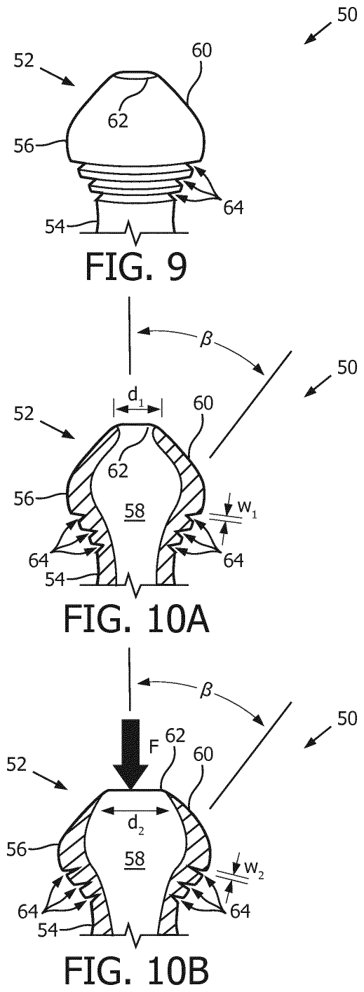

FIGS. 9, 10A and 10B show a nasal pillow 50 in accordance with another example embodiment of the present invention. Pillow 50 is preferably formed of a soft, cushiony, elastomeric material, such as silicone, appropriately soft thermoplastic elastomers, closed cell foam, thin materials, or any combination of suitable materials. Pillow 50 has a first end portion 52, a second end portion 54 disposed opposite first end portion 52, and a wall portion 56 extending between first end portion 52 and second end portion 54. A passage 58 is defined within pillow 50, generally by wall portion 56, which extends between first end portion 52 and second end portion 54 through which a flow of breathing gas produced by a pressure/flow generating system (such as generating system 12 of FIG. 1) passes from second end portion 54 to first end portion 52 and then to the airway of the patient. First end portion 52 includes a sealing flap 60 which generally defines a patient opening 62 in first end portion 52. Sealing flap 60 is structured to sealingly engage a surface of a patient about an airway of the patient (e.g., flap 60 sealingly engages about a nare of a patient). Second end portion 54 is adapted to receive the flow of breathing gas produced by a pressure flow generating system either via an intermediary component, such as a mask or other suitable component, or direct or indirectly via any suitable arrangement.

Continuing to refer to FIGS. 9, 10A and 10B, wall portion 56 includes a plurality of notches 64 which are positioned and structured to deform in a predetermined manner upon application of a force F (FIG. 10B), such as when pillow 50 is pressed against the nare of a patient. More particularly, notches 60 are structured to collapse from an initial position (FIG. 10A) wherein the notches 60 have a first opening width w₁, to a second position (FIG. 10B) in which the notches 60 have a second opening width w₂ which is less than first opening width w₁. Such deformation of selected portions, or the entirety, of wall portion 56 results in outward (i.e., away from passage 58) movement of the region of wall portion 56 near first end portion 52, such as shown by the increase in angle β between FIGS. 10A and 10B, thus increasing the diameter of patient opening 62 from a first diameter d₁ (FIG. 10A) to a greater second diameter d₂ (FIG. 10B). Such movement thus produces a tensioning force in at least a portion of sealing flap 60.

In the example illustrated in FIGS. 9, 10A and 10B, each of notches 64 are positioned generally circumferentially about pillow 50 and parallel to at least a portion of first end portion 52 and/or sealing flap 60, however, it is to be appreciated that such notches 64 may be positioned in other orientations without varying from the scope of the present invention. It is also to be appreciated that the quantity of such notches 64 may also be varied without varying from the scope of the present invention.

From the foregoing description it is to be appreciated that the invention improves seal between a cushion and a patient's face by actively avoiding the formation of buckles in the sealing flap. As the cushion is compressed on a patient's face, the wall portion of the cushion deforms in a predetermined manner which produces a tensile force in the sealing flap. The presence of such tensile force(s) works to actively prevent the formation of buckles in the sealing flap and thus improve the seal between the cushion and the patient.

It is to be appreciated that the present invention is not intended to be limited to the mask or cushion shapes described herein but instead may be employed with masks and cushions of various other shapes or designs and particularly may be applied to masks which encompass any or all airways (e.g., full face masks, nasal masks, nasal pillows, oral masks).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion for use in a system providing a flow of breathing gas to the airway of a patient, the cushion comprising:
a first end portion having a sealing flap which is structured to sealingly engage a surface of the patient about an airway of the patient;
a second end portion adapted to receive the flow of breathing gas; and
a wall portion extending between the first end portion and the second end portion and defining a passage extending between the first portion and the second portion, the passage being structured to communicate the flow of breathing gas from the second end portion to the first end portion, wherein the wall portion includes a notch which is positioned and structured to deform in a predetermined manner such that an angle (ϕ) which the wall portion makes with a longitudinal axis of the passage increases from a first value to a second value upon deformation of the notch, wherein said deformation of the notch produces a tensioning force in at least a portion of the sealing flap.

2. The cushion of claim 1, wherein the notch extends into the wall portion from an outer surface of the wall portion disposed opposite the passage.

3. The cushion of claim 2, wherein the wall portion comprises an inner surface facing the passage, and wherein the inner surface is generally uninterrupted and smooth.

4. (canceled)

5. The cushion of claim 1, wherein the notch is disposed generally parallel to the sealing flap or generally perpendicular to the sealing flap.

6. The cushion of claim 1, wherein the notch is disposed generally parallel to the sealing flap or generally perpendicular to the sealing flap; and wherein the wall portion further comprises a second notch disposed generally parallel to the sealing flap or generally perpendicular to the sealing flap.

7. The cushion of claim 1 wherein the notch is structured to deform from a first position in which the notch has a first opening width (w₁) to a second position in which the notch has a second opening width (w₂) which is less than the first opening width.

8. The cushion of claim 1 wherein the notch is structured to deform from a first position in which the notch has a first opening width (w₁) to a second position in which the notch has a second opening width (w₂) which is greater than the first opening width.

9. A cushion for use in a system providing a flow of breathing gas to the airway of a patient, the cushion comprising:
a first end portion having a sealing flap which is structured to sealingly engage a surface of the patient about an airway of the patient, the sealing flap defining a patient opening therein;
a second end portion adapted to receive the flow of breathing gas; and
a wall portion extending between the first end portion and the second end portion and defining a passage extending between the first portion and the second portion, the passage being structured to communicate the flow of breathing gas from the second end portion to the first end portion,
wherein the wall portion includes a notch which is positioned and structured to deform in a predetermined manner which increases the perimeter of the patient opening.

10. The cushion of claim 9, wherein the notch is disposed generally parallel to the sealing flap or disposed generally perpendicular to the sealing flap.

11. The cushion of claim 10, wherein the notch is disposed generally parallel to the sealing flap or generally perpendicular to the sealing flap and wherein the wall portion includes a second notch disposed generally parallel to the sealing flap or generally perpendicular to the sealing flap.

12. The cushion of claim 9 wherein the notch is structured to deform from a first position in which the notch has a first opening width (w₁) to a second position in which the notch has a second opening width (w₂) which is less than the first opening width.

13. The cushion of claim 9 wherein the notch is structured to deform from a first position in which the notch has a first opening width (w₁) to a second position in which the notch has a second opening width (w₂) which is greater than the first opening width.

14. A system for providing a pressurized flow of gas to the airway of a patient, the system comprising:
a pressure/flow generating system; and
a cushion comprising:
a first end portion having a sealing flap which is structured to sealingly engage a surface of the patient about an airway of the patient;
a second end portion coupled to the pressure/flow generating system; and
a wall portion extending between the first end portion and the second end portion and defining a passage extending between the first portion and the second portion, the passage being structured to communicate the flow of breathing gas from the second end portion to the first end portion, wherein the wall portion includes a notch which is positioned and structured to deform in a predetermined manner such that an angle ϕ which wall portion makes with a longitudinal axis the passage 36 increases from a first value to a second value upon deformation of the notch, wherein said deformation of the notch produces a tensioning force in at least a portion of the sealing flap.

15. The system of claim 14, wherein the wall portion comprises an inner surface facing the passage and an outer surface disposed facing away from the passage;
wherein the notch extends into the wall portion from the outer surface; and wherein the inner surface is generally uninterrupted and smooth.
